(19) United States
(12) Patent Application Publication
(10) Pub. No.: US 2018/0015006 A1
(43) Pub. Date: Jan. 18, 2018

(54) POWDER AND METHOD FOR CLEANING TEETH

(71) Applicant: FERTON HOLDING S.A., DELÉMONT (CH)

(72) Inventors: Marcel DONNET, Saint Jean de Gondville (FR); Sauvageot-Maxit KARINE, Cuvat (FR)

(21) Appl. No.: 15/548,620

(22) PCT Filed: Jan. 18, 2016
(86) PCT No.: PCT/EP2016/050892

§ 371 Date: Aug. 3, 2017

(30) Foreign Application Priority Data
Feb. 3, 2015 (DE) 10 2015 201 871.7

(51) Int. Cl. A61K 8/02; A61K 8/44; A61K 8/19; A61C 3/025; A61K 8/34; A61Q 11/00; A61K 8/60

(52) U.S. Cl. CPC A61K 8/022; A61Q 11/00; A61K 8/44; A61K 8/0241; A61K 8/60; A61C 3/025; A61K 8/345; A61K 8/19; A61K 2800/28; A61K 2800/412

(57) ABSTRACT

Powder for use in a powder jet device for cleaning a tooth surface by spraying powder, wherein the powder is sprayed onto the tooth surface together with air to dean the tooth surface, characterized in that the powder contains sucralose.

The invention relates to a powder for use in a powder jet device for cleaning a surface of a tooth by powder blasting wherein the powder is sprayed onto the tooth's surface, together with a gaseous carrier medium, in particular air and optionally a fluid, such as water, for cleaning the tooth's surface. The invention additionally relates to a method for cleaning teeth by using a powder jet device by means of which a powder together with a gaseous carrier medium, in particular air and optionally a fluid, such as water, is sprayed onto a tooth surface.

Such dental cleaning powders, i.e., dental powders and corresponding methods for cleaning teeth are known in the state of the art. Here, the powder is sprayed together with a gaseous carrier medium, usually air, onto the tooth's surface, thereby achieving a particularly efficient cleaning of the tooth's surface. Additionally or as an alternative to a gaseous carrier medium, also a liquid carrier medium, for example, water, may in principle also be used.

A dental cleaning powder of the aforementioned type is described in e.g. DE 200 09 665 U1. The powder contains a basic powder of sodium bicarbonate, alternatively aluminum oxide or dolomite and additional active ingredients which have for example an anti-microbial or disinfectant flavourful effect or an effect which contributes to the remineralization of the teeth.

Another powder for use in a powder jet device is described in EP 2 228 175 A1. This powder is based on alditols, in particular mannitol and/or erythritol.

DE 29 30 836 A1 describes a method for cleaning teeth in which the particles are directed by means of a stream of gas onto the surface of a tooth to be cleaned and at the same time a water stream surrounding the gas stream is also directed on this surface.

DE 100 26 718 A1 describes a suitable dental abrasive jet device for a dental powder, for example, comprised of sodium bicarbonate.

The object of the invention is to provide a powder for cleaning teeth and a method for cleaning teeth which will permit efficient and minimally unpleasant cleaning of teeth. The invention is also based on the object of making available a dental cleaning powder having an improved storage capacity and an improved distribution in the gas stream, i.e., a powder which can be stored for a longer period of time and which can be fluidized better.

This object is achieved according to the invention by a powder (dental cleaning powder, dental powder) having the features of claim 1, a method for cleaning teeth having the features of claim 8 and the use according to claim 11. Preferred embodiments of the invention are the subject matter of the dependent claims as well as the following description.

The powder according to the invention for cleaning teeth is characterized in that it contains sucralose. This method is characterized accordingly by the fact that the powder used contains sucralose. This powder is used according to the invention as an agent for processing by means of a stream of powder, in particular by means of powder jet cleaning, of tooth surfaces, in particular of dentin.

The powder is suitable and is provided for being sprayed in powder form together with a gaseous carrier medium, preferably air, onto the tooth surface (supragingivally or subgingivally). The powder is provided and is suitable in particular for use in an abrasive spray device (powder jet device, pressure spray device). Here, an abrasive and/or cleaning treatment of tooth surfaces is carried out through the impact of the particles of powder in the stream of air. A powder jet device comprises a powder chamber in which the powder is fluidized by means of a stream of air that is created. The resulting air-powder mixture is then directed on the tooth surface to be cleaned by means of a line and an outlet nozzle. Deposits on the tooth surface are removed in doing so as a result of the impact of the particles with the tooth surface under pressure.

Sucralose has several advantageous effects in the dental cleaning powder, i.e., dental powder. Sucralose primarily prevents binding of moisture in the powder so that the powder remains suitable for storage for a longer period of time. In addition it has been found that an improvement in the taste of the powder can be achieved by using sucralose in comparison with sugar or saccharin, for example. Finally, sucralose improves the graininess of the powder, i.e., it fluidizes better in the powder chamber and also becomes distributed better in the stream of gas. The cleaning effect and/or abrasive effect on the teeth is/are more uniform and there is no unwanted pulsation of the powder/air stream.

The use of sucralose in the dental cleaning powder therefore permits the use of a basic powder comprised of abrasive particles, which tend to absorb water (are hygroscopic) and/or have an unpleasant taste and/or form clumps or tend to form clumps. In particular the properties of a dental cleaning powder based on a water-soluble base powder, for example, sodium hydrogen carbonate (sodium bicarbonate) or improved. On one hand, it is desirable for the powder to dissolve in the patient's mouth after it strikes the tooth's surface. On the other hand, good storability is a goal. By adding sucralose to the dental cleaning powder, these two properties can be combined with one another advantageously.

In addition, the salty taste of sodium bicarbonate (sodium hydrogen carbonate), for example, which is usually perceived as unpleasant, can be covered or masked much better by sucralose than by sugar or other sweeteners, such as saccharin, for example. Saccharin, which was previously used in most cases, has the disadvantage that an overdose leads to a bitter taste, while a “normal” dose does not adequately mask the unpleasant taste of sodium bicarbonate. In addition, when most sweeteners are combined with salt, the result is an unpleasant taste. However, sucralose does not have these disadvantages.

Experiments have shown that the pourability of the basic powder declines with a longer storage time due to the uptake of moisture. Addition of conventional sweeteners, such as saccharin, also promotes the moisture uptake by the powder and is therefore at least counterproductive for this property of the powder. However, powder containing sucralose as an additive does not have these negative properties. It remains pourable for a much longer period of time. This is important in particular for use in powder jet devices, because the fluidization of the powder in the powder chamber leads to satisfactory results only when a sufficiently pourable powder is used.

The present invention thus makes available a dental powder which has a pleasant taste, can be stored well and is also more granular.

The powder preferably contains a basic powder, in particular at least 90% by weight of an abrasive basic powder for tooth cleaning. The basic powder contains and/or consists of particles, which act as an abrasive for tooth cleaning. The amount of the basic powder by weight relative to the total powder is preferably at least 90% by weight, in particular at least 95% by weight, especially preferably at least 97% by weight. The basic powder contains in particular sodium hydrogen carbonate (sodium bicarbonate) and/or an alditol, such as erythritol and/or glycine, for example. Other substances that are used as the basic powder are also suitable for dental powder jet devices, for example, calcium carbonate or aluminum trihydroxide. The basic powder preferably consists of at least one substance selected from the group of sodium hydrogen carbonate, an alditol such as erythritol and/or glycine.

In a preferred embodiment of the invention, the powder contains at least 90% by weight, preferably at least 95% by weight, especially preferably at least 97% by weight sodium hydrogen carbonate, erythritol and/or glycine. The aforementioned substances, i.e., the basic powder, have an abrasive action and thus form the agent for tooth cleaning by being sprayed onto the tooth surface. Additives may optionally be mixed with the basic powder. However, fundamentally any other substances such as other alditols may also be used as the basic powder.

With regard to the water binding and taste properties, it has also proven advantageous for the powder to contain 0.1% by weight to 3.0% by weight, preferably 0.1% by weight to 1.8% by weight, especially preferably 0.2% by weight to 1.5% by weight sucralose.

The powder preferably additionally contains amorphous silica, preferably approximately 0.1% by weight to 5% by weight, in particular 0.2% by weight to 3% by weight, especially preferably 0.3% by weight to 2.5% by weight. Silica particles improve the pourability of the powder and reduce the moisture uptake.

In addition, it is preferable for the powder to contain at least one flavoring. The flavoring may fundamentally be selected at will and may contain mint or menthol. The flavoring is preferably coordinated with the taste of the basic powder (e.g., sodium hydrogen carbonate) and/or the sucralose. The flavoring may be present in a particularly low concentration because the flavoring does not have to cover an unpleasant taste of the basic powder, for example, in particular if it is sodium hydrogen carbonate. According to the invention, sucralose assumes this function in the dental cleaning powder. The powder preferably contains 0.05% by weight to 1.5% by weight of a flavoring, especially preferably 0.1% by weight to 1.2% by weight.

According to the invention, it is preferable in particular for the sucralose to be present in the powder in a concentration such that the taste of the basic powder, in particular sodium hydrogen carbonate, is covered and/or reduced to below the limit of perception.

For a gentle and nevertheless efficient tooth cleaning, it is preferable for the particles of the basic powder to have an average grain size of 10 μm to100 μm, in particular 10 μm to 65 μm. Basically, however, grain sizes of up to 200 μm are also conceivable. The abrasive particles of the basic powder may be adapted in particular to the field of application of the dental cleaning powder that is provided. For example, a smaller grain size, in particular approximately 10 μm to 30 μm is preferred for cleaning subgingival tooth surfaces. For tooth cleaning of the supragingival tooth surfaces, a larger grain size, in particular approximately 40 μm to 65 μm, is preferred.

In another preferred embodiment of the dental cleaning powder according to the invention, the basic powder is water-soluble. In other words, the particles for cleaning the tooth surface (particles of the basic powder and/or abrasive particles) are water-soluble. Therefore, they dissolve in the patient's mouth and can thus be removed easily after fulfilling their task of tooth cleaning.

In experiments it has been found that sucralose has the special property of dissolving much more rapidly in water than other sweeteners such as saccharin. Therefore sucralose can also manifest its taste-improving properties much more quickly and can compensate for the salty taste of the readily soluble sodium hydrogen carbonate. Due to the good and rapid solubility of sucralose, even small amounts are sufficient to achieve this effect. An especially preferred combination is therefore to add sucralose to a sodium hydrogen carbonate powder. The salty taste of the sodium hydrogen carbonate powder is compensated especially rapidly and effectively by the pleasantly sweet taste of sucralose. Therefore there is no bitter aftertaste.

It is preferable for the powder to contain sucralose in powder form (in the form of a powder). The powder thus contains abrasive particles for the cleaning of the tooth surface (basic powder) and particles made essentially of sucralose (sucralose corpuscles or particles). The powder thus contains at least two different particles or types of particles, namely first cleaning particles and second sucralose particles (powder mixture). The cleaning particles and the sucralose particles are preferably each soluble in water and are coordinated with one another, in particular with regard to their surface and/or particle size and amount, so that the dissolving rate in water that is achieved is one at which the two taste directions basically compensate for one another. This may also be understood to mean that the totality of the abrasive particles (sodium hydrogen carbonate and/or sodium hydrogen carbonate powder) and the totality of the sucralose and/or sucralose particles will dissolve after approximately the same amount of time. This optimizes the taste for the patient because the taste of the dissolving cleaning particles is covered by sucralose particles dissolving at the same time and/or in parallel with this.

Sucralose corpuscles or with an average size between 5 and 40 μm have been found to be suitable and sucralose particles with an average particle size between 10 and 25 μm have been found to be especially suitable. However, other particle sizes can also be used.

In another preferred embodiment, the powder contains abrasive particles for cleaning the tooth surface (basic powder), wherein the sucralose is present in the abrasive particles (basic powder). This could be achieved, for example, by agglomeration, coating or precipitation, by using spray drying or in a fluidized bed. Other known methods or processes for combining the two substances are also conceivable.

It is provided in a particularly preferred embodiment of the method according to the invention for tooth cleaning that the powder contains water-soluble abrasive particles; that the water-soluble abrasive particles contain sodium hydrogen carbonate in particular; that a powder-air mixture and a liquid stream are sprayed separately onto a tooth surface; that the powder contains sucralose, and that the sucralose is dissolved at the same time and in particular at the same dissolving rate after striking the tooth surface. To do that, preferably a nozzle device of a powder spray device is used, having a first nozzle for the powder-air mixture and a second nozzle for the liquid spray, in particular the water spray. The second nozzle is preferably arranged around the first nozzle, in particular in a ring shape. The powder-air mixture first strikes the tooth surface in an essentially dry form. Then the powder is moved away by the stream of water and dissolved. In doing so, the sodium hydrogen carbonate and the sucralose dissolve approximately at the same time.

### PREFERRED EXEMPLARY EMBODIMENTS

The following examples provide preferred powders according to the invention.

### Exemplary Embodiment 1

Sodium hydrogen carbonate powder (particle size 40): 97.2% by weight

Amorphous silica: 2.5% by weight

Flavoring: 0.1% by weight

Sucralose: 0.2% by weight

### Exemplary Embodiment 2

Erythritol (particle size 70 μm): 95.0% by weight

Amorphous silica: 2.5% by weight

Flavoring: 1.0% by weight

Sucralose: 1.5% by weight

### Exemplary Embodiment 3

Erythritol (particle size 13 μm): 94.8% by weight

Flavoring: 1.2% by weight

Sucralose: 1.5% by weight

### Exemplary Embodiment 4

Glycine (particle size 20 μm): 95.0% by weight

Amorphous silica: 2.5% by weight

Flavoring: 1.0% by weight

Sucralose: 1.5% by weight

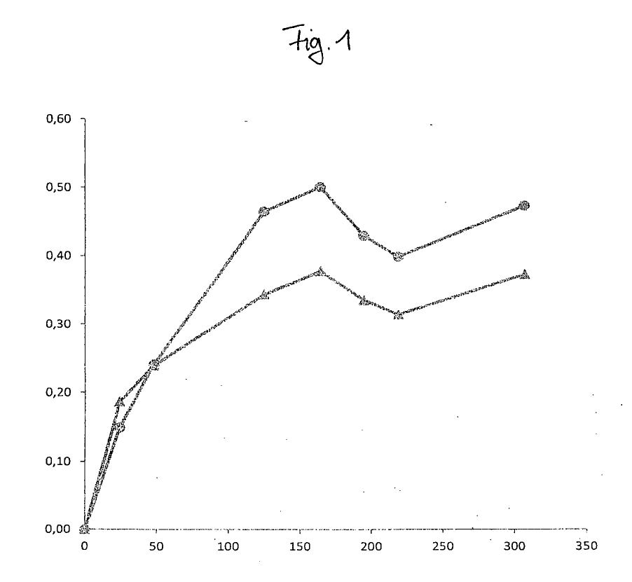

The invention is described further below with reference to the single FIGURE (FIG. 1).

FIG. 1 shows a measurement series for moisture uptake by a powder according to the invention and/or a powder mixture (graph with triangular measurement points) in comparison with a reference powder (graph with round measurement points). The powder according to the invention is the composition according to Exemplary Embodiment 1. In the reference powder, the sucralose was replaced by saccharin. Both powders had an average grain size of 40 μm. These experiments were carried out with open bottles a temperature of 25° C. and a relative humidity of 85% in a climate chamber. FIG. 1 shows the time in hours on the abscissa and the water content in weight percent on the ordinate.

After conclusion of the measurements, the mixture mixed with sucralose still had a powdery behavior while the known powdered mixture with saccharin had assumed a pasty consistency. The reference powder was therefore no longer to be used as a spray means.

This measurement series shows in particular that there is a significant difference between these two powders after approximately 80 hours. The maximum water uptake by the powder according to the invention was approximately 0.38% by weight.

However, the maximum water uptake by the known powder was approximately 0.5% by weight.

It has thus been demonstrated that adding sucralose to the mixture would yield a powder and/or a powder mixture with a much lower water uptake due to the sucralose content.

1.-14. (canceled)

15. A powder for use in a powder jet device for cleaning a tooth surface by powder spraying, wherein the powder for cleaning the tooth surface is sprayed onto the tooth surface together with a gaseous carrier medium, the powder comprising sucralose and at least 90% by weight sodium hydrogen carbonate, erythritol and/or glycine.

16. The powder according to claim 15, wherein the powder contains 0.1% by weight to 3.0% by weight sucralose.

17. The powder according to claim 15, wherein the powder contains sucralose in powder form, wherein the sucralose particles have an average size between 5 μm and 40 μm.

18. The powder according to claim 15, wherein the powder contains sucralose in powder form, wherein the sucralose particles have an average size between 10 μm and 25 μm.

19. The powder according to claim 15, wherein the powder contains amorphous silica.

20. The powder according to claim 15, wherein the powder contains at least one flavoring.

21. The powder according to claim 15, wherein the powder contains a basic powder, whose particles have an average grain size of 10 μm to 75 μm.

22. The powder according to claim 15, wherein the powder contains a basic powder, which is water-soluble.

23. A method for cleaning teeth by using a powder spray device, comprising spraying a powder onto a tooth surface together with a gaseous carrier medium according to claim 15, wherein the powder contains sucralose.

24. The method according to claim 23, wherein the powder contains water-soluble abrasive particles, wherein the water-soluble abrasive particles contain sodium hydrogen carbonate in particular, wherein a powder-air mixture and a fluid spray are sprayed separately onto a tooth surface, wherein the powder contains sucralose, and wherein the sucralose dissolves at the same time with and in particular at the same dissolving rate after striking the tooth surface.

25. The powder according to claim 15, further comprising additional finely divided substances including at least one of silica gel, bleaching agents, analgesics and/or bactericides as the agents for powder spray treatment, wherein for the powder is configured for powder spray cleaning dentin.

26. The powder according to claim 25, wherein the agent is configured to be applied to the tooth surfaces to be treated together with air and water by means of a powder spray device.
